(19) United States
(12) Patent Application Publication
(10) Pub. No.: US 2025/0252290 A1
(43) Pub. Date: Aug. 7, 2025

(54) System and Method for Designing Stimuli for Neuromodulation

(71) Applicant: CARNEGIE MELLON UNIVERSITY, Pittsburgh, PA (US)

(72) Inventors: Pulkit Grover, Pittsburgh, PA (US); Chaitanya Goswami, Pittsburgh, PA (US)

(73) Assignee: CARNEGIE MELLON UNIVERSITY, Pittsburgh, PA (US)

(21) Appl. No.: 18/727,377

(22) PCT Filed: Oct. 30, 2023
(86) PCT No.: PCT/US2023/078171

§ 371 Date: Jul. 9, 2024

Related U.S. Application Data
(60) Provisional application No. 63/420,725, filed on Oct. 31, 2022.

(51) Int. Cl. G06N 3/045

(52) U.S. Cl. CPC G06N 3/045

(57) ABSTRACT

Disclosed herein is a novel pseudoinverse estimation system and method that adapts regression techniques to directly estimate one or more pseudoinverses of a neuromodulation pathway, thereby circumventing the need of inverting an estimated forward model. This is accomplished by the learning of a restricted domain that restricts the potential stimuli required to produce a desired neuro response.

### RELATED APPLICATIONS

This application claims the benefit of U.S. Provisional Patent Application No. 63/420,725 filed Oct. 31, 2022, the contents of which are incorporated herein in its entirety.

### GOVERNMENT INTEREST

This invention was made with U.S. Government support under contract 46436.8.1.1990701, awarded by DARPA. The U.S. government has certain rights in this invention.

### BACKGROUND OF THE INVENTION

Neuromodulation refers to altering neural activity through the targeted delivery of a stimulus (e.g., electrical, chemical, ultrasound), and is one of the fastest growing areas of medicine, impacting millions of patients. Many neurological disorders and diseases are a result of atypical neural activity in the brain and can be treated by providing appropriate stimuli which can “correct” the atypical neural activity.

In experiments, controlling the neural activity through stimuli has shown promise in treating Parkinsonian symptoms, stroke rehabilitation, regulating depression, etc. The ability to systematically design stimuli that produce a desired neural response in the brain which corrects the atypical activity is key to treating several neurological disorders.

Typically, a stimulus is characterized by a set of parameters. For example, electrical currents (stimuli) can be injected into the brain to selectively stimulate a particular type of neuron (controlling neural activity) for treating Parkinsonian symptoms. This method has been tried in mice subjects, in which the stimulus is characterized by three parameters, namely, amplitude, frequency, and duration of the injected electrical currents.

A common way to mathematically model the relation between the parameters of stimuli and the neural activity/response is through a “forward mapping/function” that takes as input the parameters and outputs the neural response. The problem of designing a stimulus that produced the desired neural response is then reduced to inverting the forward mapping. Correspondingly, the set of stimulus parameters can be obtained by plugging in the desired neural response as an input to the inverse.

Broadly, there are three major challenges in estimating the inverse of the forward mapping. First, as the forward mapping depends upon the parameters being explored, for novel parameters, the forward mapping is generally unknown and needs to be estimated from the data. Second, in most cases of interest, multiple sets of parameter values characterizing a stimulus lead to the same neural response. For example, in electrical stimulation of the brain, many stimuli produce the same neural firing rate and consequently, the same neural response. This implies that the forward mapping in many cases is many-to-one, and hence, non-invertible. Therefore, instead of estimating an inverse, a pseudoinverse of the forward mapping is estimated. Third, the amount of data available in such healthcare settings is limited, and in general, the data collection process is quite expensive.

It would be desirable to be able to estimate the pseudoinverse in a data-efficient manner. There has been a significant interest in designing stimuli using data-driven method through some form of pseudoinverse estimation. A main advantage of data-driven approaches over traditional stimulus design approaches is that they allow end users (e.g., clinicians or neuroscientists) to explore a larger number of parameters as compared to traditional approaches. The ability to explore a large number of parameters allows the possibility of discovering novel stimuli that can produce neural responses which have clinical relevance.

Broadly, two methodologies have been explored for estimating the pseudoinverse for the purpose of stimulus design. One is to use conditional density estimation (CDE) methods to learn the conditional density of waveform parameters conditioned on firing rates, and then use the conditional mode as the pseudoinverse. The other is to estimate the forward mapping (e.g., using a deep neural network) and then numerically inverting it. Briefly, CDE-based methods are known to require more data than their regression counterparts, and numerical inversion approaches suffer because numerical inversion blows up even small errors in forward models.

### SUMMARY OF THE INVENTION

Disclosed herein is a novel pseudoinverse estimation system and method that addresses the shortcomings of the two methodologies mentioned above. Specifically, the disclosed method adapts regression techniques to, in one embodiment, directly estimate a pseudoinverse, thereby circumventing the need of inverting an estimated forward model, while still requiring less data than CDE methods (because regression techniques typically require less data than their equivalent CDE counterpart). In a second embodiment, an ensemble of pseudoinverses is estimated.

The key insight of the disclosed method is that a non-invertible function can still be inverted over a restricted domain. If such a restricted domain is known a priori, the inverse mapping can be estimated using regression methods. The disclosed method jointly learns a restricted domain and the inverse mapping over it. A weighted L2 loss is utilized, where the weights are also learned from data. On convergence, the weights approximate the indicator function over the restricted domain, effectively learning it.

When compared with other methods, for example, Masked Autoregressive Flows (MAF) and Mixture Density Networks (MDN), the disclosed method outperforms all the other methods when the size of dataset is small.

### BRIEF DESCRIPTION OF THE DRAWINGS

FIG. 1 are graphs showing the normalized mean absolute error (NMAE) of the disclosed method versus various prior art data-driven methods discussed herein on toy mappings.

FIG. 2 are graphs visualizing pseudoinverses for toy mappings.

FIG. 3 show graphs showing the NMAE across both neuron models with different training datasets sizes constructed from bio-physical computational neuron models.

FIG. 4 is a schematic representation of the waveforms used in the simulation study.

FIG. 5 is a dataflow diagram of the process.

FIG. 6 is a flowchart of the process.

### DETAILED DESCRIPTION

Disclosed herein is a method for directly estimating a pseudoinverse neural mapping to affect a desired neural response by restricting the domain over which the forward mapping from the stimulus to the neural response is inverted.

Assume that each stimulus is characterized by n-different parameters, denoted as {θ₁}ᵢ₌₁ⁿ, where θᵢ∈ [symbol] R. Define θ=[θ₁, θ₂, . . . , θₙ]^{T}∈ [symbol] ⁿ as the collection of all the n parameters. Θ⊂ [symbol] ⁿ denotes the collection of all allowed stimuli. Let the number of neural responses of interest be m (e.g., for selective stimulation between two neuron types, m=2). Define r=[r₁,r₂, . . . rₘ]^{T}∈R. Let [symbol] be the collection of all distinct neural responses produced by all the stimuli θ∈Θ.

To illustrate the notation further, consider an example in which the goal is to selectively stimulate a single neuron type without stimulating another neuron type for treating Parkinsonian symptoms. The stimuli of electrical currents is characterized by 3 parameters which are the amplitude, frequency and duration of the currents. So, in this example the parameters would be θ₁, θ₂, θ₃ respectively denoting the amplitude, frequency and duration of the electrical currents, and θ=[θ₁, θ₂, θ₃]^{T}∈ [symbol] ³. The relevant neural response in the same example are the firing rates of the different neuron types, so r=[r₁,r₂]^{T}, where r₁ and r₂ are the firing rates of the two neuron types. For finding a stimulus that attains a desired neural response, assume access to a dataset consisting of only [symbol] =[θᵢ, rᵢ]ᵢ₌₁^{N} formed by N stimulus-response pairs.

Problem Statement—Given a dataset [symbol] where {θᵢ}ᵢ₌₁^{N} are independent and identically distributed samples from a distribution [symbol] (θ) on Θ, and rᵢ is the neural response generated by the stimulus characterized by θᵢ, the goal is to design/find a parameter θ_{des}∈ [symbol] such that the stimulus corresponding to θ_{des} can elicit (close to) a desired (user-specified) neural response r_{des}∈ [symbol] .

Note that access is only allowed to the fixed dataset [symbol] . An important design parameter is choosing the set of allowed stimuli (i.e., Θ). This is decided a priori by the user, typically based on domain knowledge, (e.g., choose amplitude, frequency, and duration of the electrical waveforms as parameters of stimuli). Herein, it is assumed that that an appropriate Θ has already been chosen.

Denote the forward mapping from the stimulus parameter space Θ to the neural response space [symbol] as g:Θ→ [symbol] . For many-to-one functions such as g, a natural definition of a pseudoinverse is a mapping g⁻¹: [symbol] →Θᵢₙᵥ, where Θᵢₙᵥ⊆Θ is a restricted domain such that g(g⁻¹(r))=r†r∈ [symbol] . For example, cos: [symbol] →[−1,1] is many-to-one and not invertible but restricting the domain of cos to [0, 7] helps define the familiar pseudoinverse: cos⁻¹:[−1,1]→[0,π]. Note that, for a pseudoinverse, equality in the other direction, g⁻¹(g(θ))=θ∀θ∈Θ is not necessarily true, because the domain of g⁻¹ is θᵢₙᵥ (a subset of 0). A function can have multiple pseudoinverses (e.g., cos(·), with θᵢₙᵥ=[0+2nπ, π+2nπ], has infinitely many pseudoinverses, one for each n∈N. For the goal of the method disclosed herein, estimating any one pseudoinverse suffices. The estimated pseudoinverse of g is denoted as [symbol] herein.

The Method—The disclosed method estimates a pseudoinverse by exploiting the insights that many-to-one functions can be inverted over an appropriately restricted domain. If such a restricted domain Θᵢₙᵥ was known a priori, then a restricted dataset can be created by excluding all data points (θᵢ,rᵢ) where θᵢ∉Θᵢₙᵥ from the dataset [symbol] . Because g is invertible over Θᵢₙᵥ, any traditional regression technique applied to this restricted dataset would yield a pseudoinverse corresponding to Θᵢₙᵥ. Formally, a pseudoinverse on Θᵢₙᵥ can be estimated as:

$$= arg {min}_{f ∈ ℱ} \frac{1}{N} {\sum }_{i = 1}^{N} 𝕀 [ {θ}_{i} ∈ {Θ}_{inv} ] {‖ f ( {r}_{i} ) - {θ}_{i} ‖}_{2}^{2} \qquad ( 1 )$$

where:
□(·) is the indicator function; and
[symbol] is the family of functions being considered for regression.

The challenge, as outlined above, is that only the dataset (and not the forward mapping) can be accessed, so Θᵢₙᵥ is not known apriori. To address this, the disclosed method jointly estimates both a restricted domain and the corresponding pseudoinverse as follows:

$$, {\{ \hat{w} ( {θ}_{i} ) \}}_{i = 1}^{N} = arg {min}_{\begin{matrix}f ∈ ℱ , \\ w ( {θ}_{i} ) \geq 0\end{matrix}} {\sum }_{i = 1}^{N} {\underbrace{w ( {θ}_{i} ) {‖ f ( {r}_{i} ) - {θ}_{i} ‖}_{2}^{2}}}_{Loss} + {\underbrace{β {\sum }_{i = 1}^{N} {w}^{2} ( {θ}_{i} )}}_{Regularizer} , \\ s . t \frac{1}{N} {\sum }_{i = 1}^{N} w ( {θ}_{i} ) = 1 \qquad ( 2 )$$

where:
β∈ [symbol] is a hyper-parameter.

The first sum in Eq. (2) represents the loss, and the second sum in Eq. (2) is a regularizer. This optimization formulation follows the philosophy of Eq. (1), approximating □[θᵢ∈Θᵢₙᵥ] in Eq. (1) by ŵ(θᵢ) which are learned jointly with [symbol] .

How does the optimization of Eq. (2) incentivize learning of a restricted domain? If only parameters belonging to a restricted domain have non-zero weights (ŵ(θᵢ)), the loss term would be low because the corresponding inverse mapping can be estimated accurately. Hence, the loss term encourages the learning of weights that are non-zero only for some restricted domain over which g is invertible.

It is desirable that an estimate of the pseudoinverse [symbol] has its domain as the entire [symbol] , or at least as large a subset as possible, so it can provide a neural stimuli parameter for as many neural responses as possible. This implies that, for the disclosed method to estimate a pseudoinverse, the image of the restricted domain learned by it should be as large as possible. This condition is not ensured by the loss term in Eq. (2), since it is small for any restricted domain (e.g., consider two restricted domains [0,1] and [0, π] of cos(·). [0, π] is more desirable here. The loss term in Eq. (2) is small for both domains and is unable to discriminate between them).

To encourage the disclosed method to learn a restricted domain corresponding to a large response range/space, we use the following observation: If Θₘₐₓ is the restricted domain having the largest size (measured by its total probability under p(B)), then it's corresponding image/response space also has the largest size.

Hence, by learning the largest restricted domain we ensure we have the largest response space. This observation is incorporated in the regularizer and the constraint in Eq. (2) to encourage the method to learn as large a restricted domain as possible. To see this, analyze the following optimization that distills the effect of the regularizer for distinguishing among restricted domains over which g is invertible (the first term in Eq. (2) is low for such domains):

$${\{ {w}_{i}^{*} \}}_{i = 1}^{N} = arg {min}_{{\{ {w}_{i} \}}_{i = 1}^{N}} {\sum }_{i = 1}^{N} {w}_{i}^{2} , s . t . \frac{1}{N} {\sum }_{i = 1}^{N} {w}_{i} = 1 , \\ {\sum }_{i = 1}^{N} 𝕀 [ {w}_{i} \neq 0 ] = K , {w}_{i} \geq 0 ∀ i \qquad ( 3 )$$

This optimization explores the behavior of the regularizer when only K out of N total weights are non-zero and has the solution: wᵢ*=N/K for any K out of N weights, and the rest are 0. The regularizer term in Eq. (2) scales approximately as ˜1/K² for K non-zero weights, which incentivizes making larger numbers of ŵ(θᵢ) to be non-zero, encouraging the consideration of as large a restricted domain as possible.

Thus, there is a careful interplay between the loss, the regularizer and the constraint in Eq. (2). The loss encourages learning non-zero weights (ŵ(θᵢ)) only over a restricted domain; the regularizer and the constraint try to make the restricted domain as large as possible. By carefully choosing the value of β, a desirable pseudoinverse can be learned.

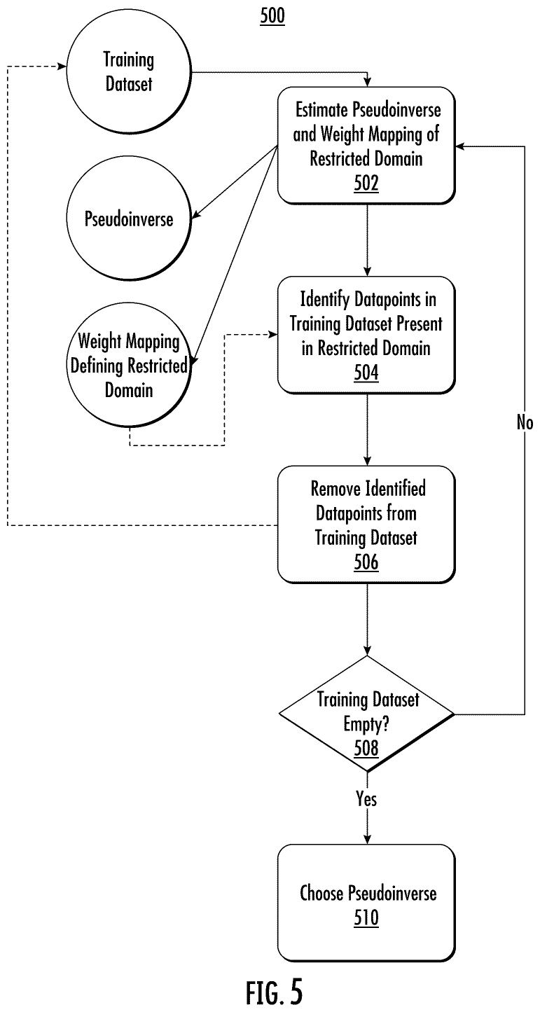

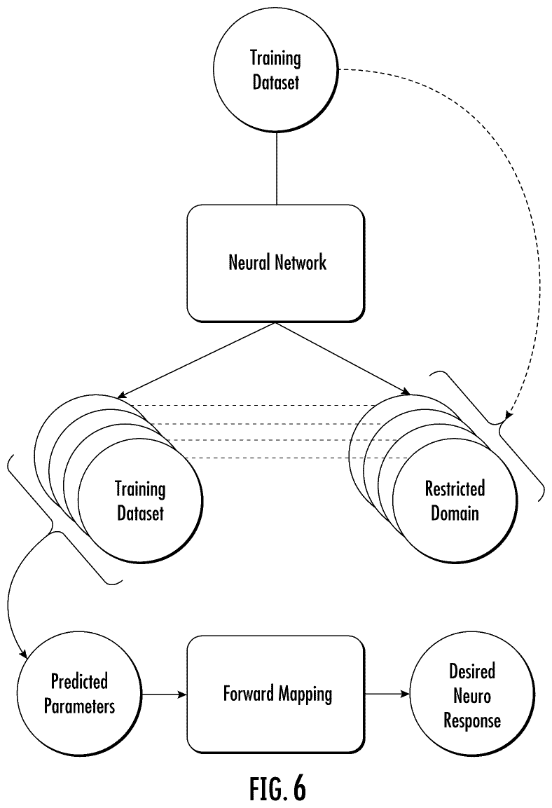

In a second embodiment of the invention, an extension of the disclosed method estimates an ensemble of pseudoinverses, instead of just one, thereby improving the performance. Process 500 of the second embodiment is shown in flowchart form in FIG. 5. The second embodiment creates an ensemble of pseudoinverses in a greedy fashion as follows: (i) the method as previous discussed is used to estimate the pseudoinverse and the corresponding weight mapping on the training dataset at step 502; (ii) at step 504, the estimated weight mapping is used to identify the datapoints in the training dataset that lie in the restricted domain (estimated in step (i)). At step 506, the identified data points are removed from the training dataset to construct a new training dataset; and (iii) This new training dataset is then again used to perform steps 502 and 504 (i.e., (i) and (ii) above, respectively). This process continues until the (remaining) training dataset becomes empty at step 506, resulting in an a plurality of pseudoinverses and corresponding restricted domains that are distinct due to step (ii). To predict the parameter vector B for some response vector \vec{r}, the pseudoinverse whose restricted domain contains the training datapoint with the response vector closest to the desired response vector output is chosen at step 510. Intuitively, this pseudoinverse would have the most confidence in predicting the right parameter. Note that no prior art method creates an ensemble of pseudoinverses in this fashion, as only the ensemble embodiment of the disclosed method explicitly estimates the restricted domain (in terms of the weight mapping) which is required for step (ii). A data flow diagram of the process is shown in FIG. 6. Although only four pseudoinverses and corresponding restricted domains are shown, as would be realized, any number of pseudoinverses and corresponding restricted domains could be produced by process 500.

Simulations—Two scenarios were simulated—toy examples and bio-physical neuron models, in which the performance of the disclosed method as well as 3 prior art data-driven methods (MAF, MDN and Naïve Inversion (NI)) were compared for estimating pseudoinverses (i.e., [symbol] (·)) In addition, a baseline approach was implemented.

BASELINE—In addition to the data-driven methods, a brute force approach, akin to the 1-Nearest Neighbor Method, is used as a baseline method for the simulation study performed. In the baseline method, the training data is used as a look-up table, where we output the parameter vector present in the training dataset whose response vector is closest to the desired response vector under the L1 distance metric.

SIMULATION WITH TOY EXAMPLES: Three different toy mappings were considered for the purpose of estimating pseudoinverses. The following models were used for generating the data from each toy mapping:

$$r = cos ( 2 π θ ) + ϵ , θ = [ 0 , 3 ] \qquad ( 4 )$$

$$r = {( {θ}^{2} - 4 )}^{2} + ϵ , θ = [ - 3 , 3 ] \qquad ( 5 )$$

$$r = {e}^{- \frac{{θ}^{2}}{2}} + ϵ , θ = [ - 3 , 3 ] \qquad ( 6 )$$

where:
ϵ˜ [symbol] (0, 0.01) is the Gaussian distribution with mean 0 and variance 0.1\hat{ }2 for cos(·) toy examples, 2.5\hat{ }2 for the polynomial(·) example and 0.2\hat{ }2 for the exponential example. In the small data regime, the disclosed method outperforms MAF, MDN and NI in estimating the pseudoinverse.

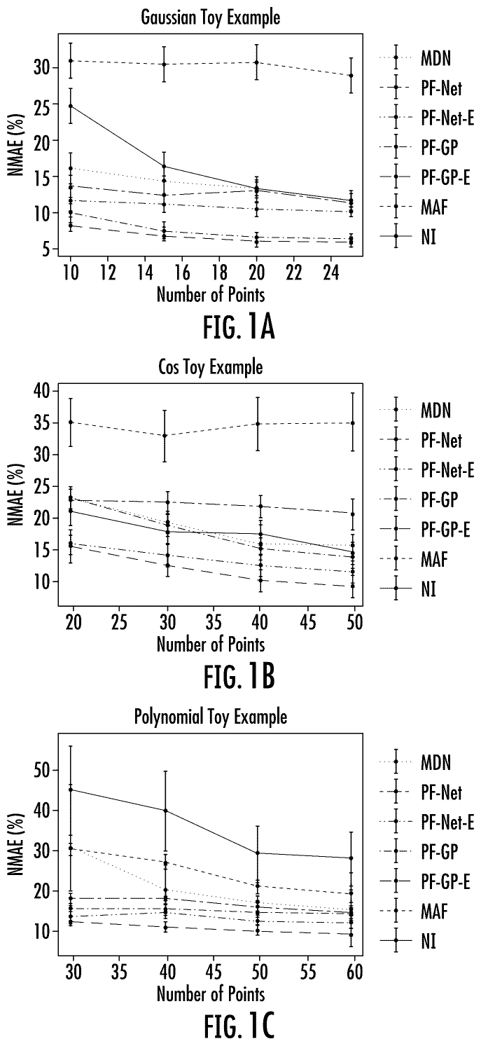

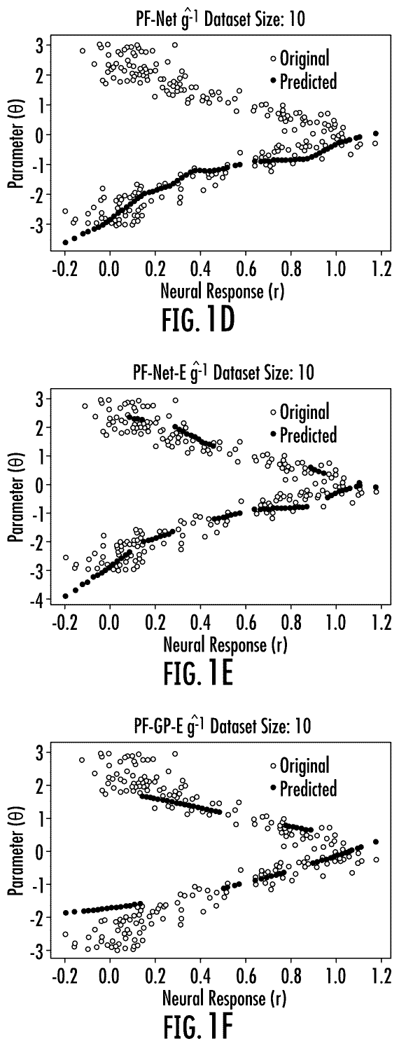

To characterize the data-efficiency of each technique, (i.e., the four variants of the disclosed method and MDN, MAF, NI) in estimating the pseudoinverses of the above toy mappings, the following study was performed: For each toy forward mapping we evaluate the NMAE at 4 different training dataset size. The resultant NMAE for all the four techniques and the three different toy mappings are shown in FIG. 1.

It can be seen a variant of the disclosed method (denoted as “PF-NET”, “PF-NET-E”, “PF-GP” or “PF-GP-E” in the FIGS. 1(a-c) has the smallest NMAE among all the competing techniques consistently across the three different toy forward mappings. This provides evidence that the method requires the least amount of data to estimate pseudoinverse with reasonable accuracy. This result extends to the more realistic simulation study of electrical stimulation of neurons below.

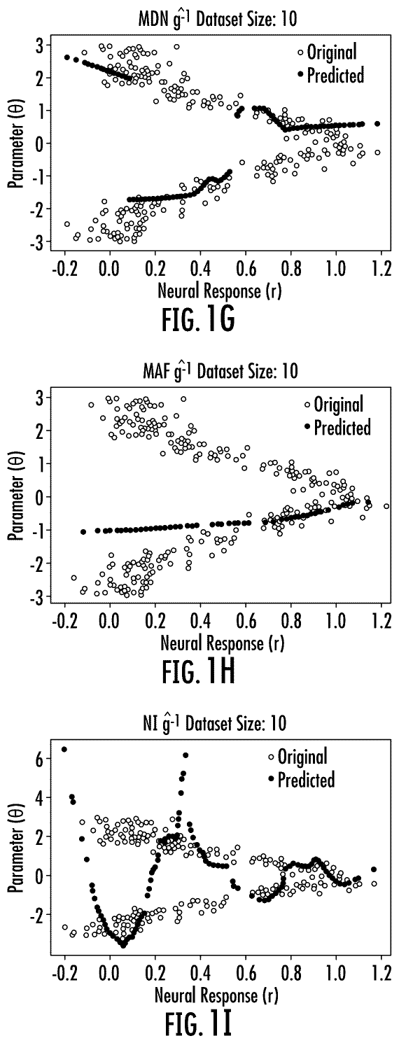

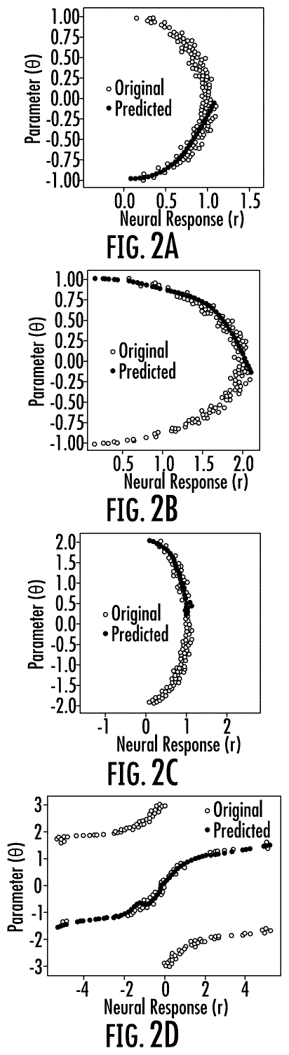

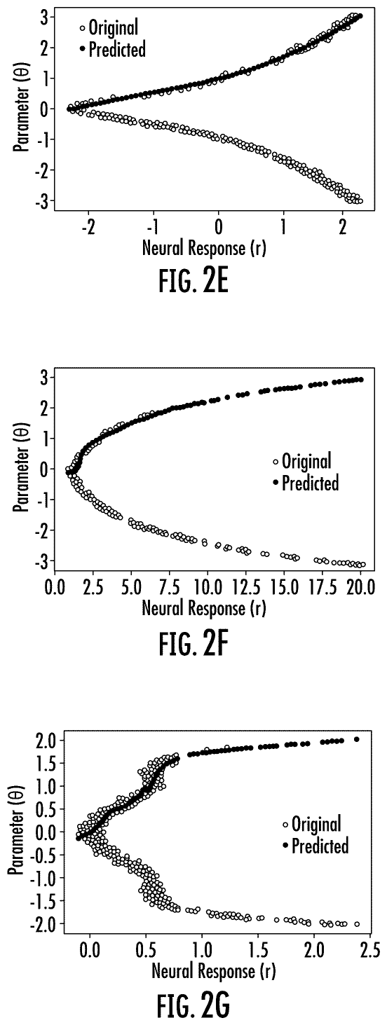

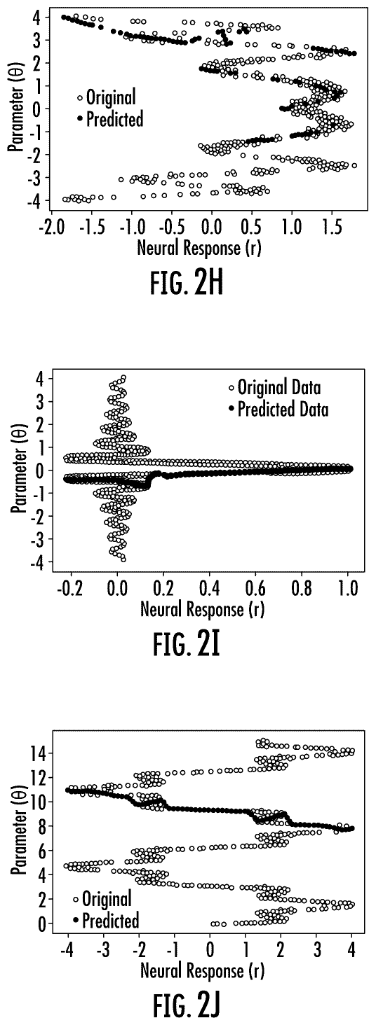

FIG. 2 shows graphs visualizing pseudoinverses estimated by the disclosed method for several non-linear functions. The corresponding forward functions for each subfigure are as follows: FIG. 2a: r=\sqrt{1−θ²}; FIG. 2b: r=2\sqrt{1−θ²}; FIG. 2c: r=\sqrt{1−(θ/2)²}; FIG. 2d: r=tan θ; FIG. 2e: r=log(0.1+x²); FIG. 2f: r=e^{|x|}, FIG. 2g: r=x²+cos(x²)−1: FIG. 2h: r=sin(|x|)+cos(x²); FIG. 2i: r=sin(πx)/(πx) and FIG. 2j: r=3 sin(θ)+sin(5θ).

SIMULATION WITH NEURON MODELS—The disclosed method and the 3 competing prior art models (MDN, MAF and NI) were applied to estimate the parameters of stimulus (electrical waveform in this particular case) for desired neural responses (firing rate of the two neuron models).

The main goal of this simulation is to show that the performance of the disclosed method is better at “small” dataset size and is comparable to other methodologies at “large” dataset size. That is, qualitatively the results obtained for toy examples continue to hold in a more realistic scenario.

The simulation evaluates the ability of electrical waveforms to stimulate a bio-physically detailed cortical neuron model of excitatory pyramidal cell (Pyr neurons) and inhibitory parvalbumin-expressing inhibitory neurons (PV neurons). The ability to modulate the activity/firing rate of excitatory and inhibitory neurons using electrical currents has clinical relevance.

The NMAE was calculated for all the data-driven approaches, as well as the baseline approach and the results are shown in FIG. 2. For disclosed method, only the results for the best performing variant across all the toy examples are shown. FIG. 2a. shows the mean of the NMAE across the two neuron types, for all the methods with different training dataset size constructed from the bio-physical computational neuron models. FIG. 2b. is a zoomed-in version of FIG. 2a showing the average NMAE for the three top-performing methods, namely the disclosed method, Baseline, and MAF. The values of NMAE at each training dataset size and for each technique was calculated across 50 different trials, and the average NMAE is reported. The color bars show the 95% confidence interval. FIG. 2c shows the relative decrease in the average NMAE of the disclosed method compared to the next-best alternative method. To quantify this relative decrease, we plot (NMAE of next best alternative NMAE of the disclosed method)/NMAE of the disclosed method×100 for every training dataset size. The particular dataset sizes investigated for the neuron model case are informed by experimental constraints in collecting datasets.

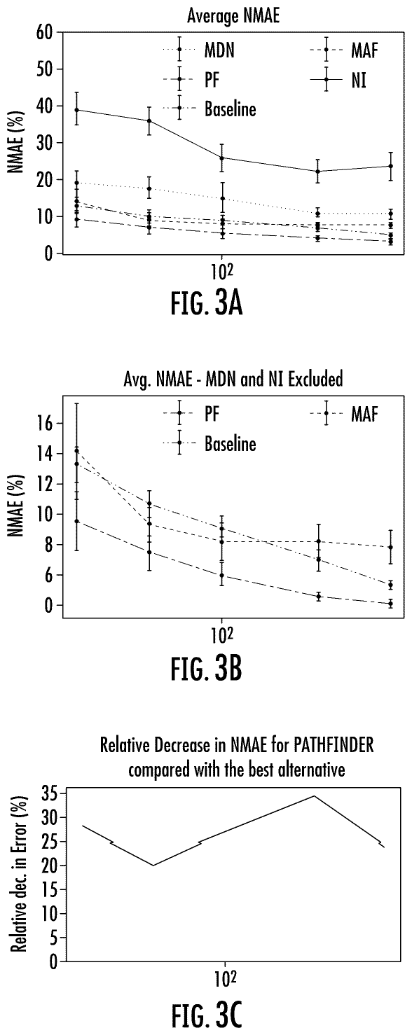

As with the toy examples, it was observed that the disclosed method significantly outperforms the other techniques for low sample size, as shown in FIGS. 3(a-c). The disclosed method has >20% less NMAE compared to the best alternative for all training dataset sizes (FIGS. 2a, 2b, and 2c). Notably, disclosed method requires only 50 datapoints to achieve the performance of the best alternative method at at 250 datapoints.

To create a dataset of size N for a toy mapping (i.e., [symbol] _{toy}={θᵢ,rᵢ}ᵢ₌₁^{N}), N samples of θ were uniformly randomly sampled from the toy forward mapping's respective domains provided in Eqs. (4-6). The corresponding {rᵢ}ᵢ₌₁^{N} for each toy mapping were then generated according to the respective models provided in Eqs. (4-6). Different values of N were considered for different toy mappings to compare performance of the disclosed method and the three prior art methods across different dataset sizes.

Bio-physical Neuron Models: It is well known that cortex consists of excitatory and inhibitory neurons, and it is hypothesized that a careful interplay between the activation of excitatory and inhibitory neurons in the cortex drives the neural activity in the cortex. Imbalance in the activation of excitatory and inhibitory neurons is linked to neurological diseases such as seizures, hence being able to modulate the activity of excitatory and inhibitory neurons is of clinical relevance. In recent years, there has been a growing interest in exploring electrical stimulation as a means to stimulate excitatory and inhibitory at desired firing rates. Controlling the firing rates of excitatory and inhibitory neurons (neural response) with electrical stimulation (stimulus) provides a good simulation setup to test the performance of the disclosed method and the three prior art methods. in a more realistic neuromodulation context.

Morphologically-realistic and bio-physically detailed multicompartment neuron models taken from the Allen Cell Type Database were used in the simulations. One neuron model is of a cortical pyramidal (Pyr; excitatory) neuron and the other is of a cortical parvalbumin-expressing (PV; inhibitory) neuron. Both neurons were simulated using the NEURON software, with the allensdk package in python.

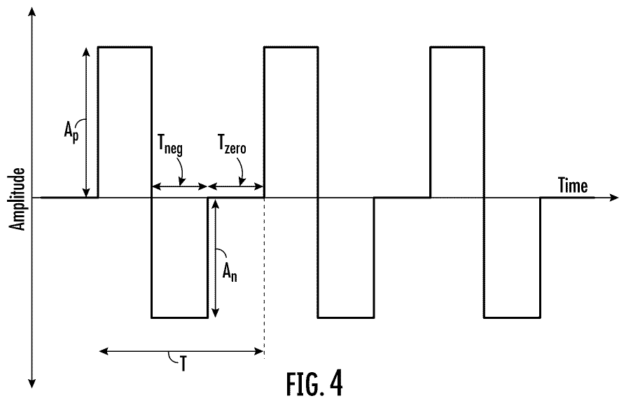

A parametric waveform family using 5 parameters is constructed: Q, Aₙ/Aₚ, T, T_{zero}/T, T_{neg}/T−T_{zero}, where Aₚ, Aₙ, T, T_{zero}, T_{neg} are as depicted in FIG. 4. Q refers to the total absolute charge of the waveform. The range of each parameter is as follows: [0.15 nC, 0.35 nC] for Q, [0.5, 2] for Aₚ/Aₙ, [0.1 ms, 500 ms] for T, [0, 0.5] for T_{zero}/T, and [0, 0.6] for T_{neg}/T−T_{zero} Each parameter is uniformly sampled from its respective range. The duration of the waveform is 1000 ms. The corresponding waveform u_{θ̅}ₙ(t) (generated from \vec{θₙ}) is injected intracellularly into the soma of each neuron model. Synaptic noise is also added to the models by adding additive white Gaussian noise with a mean of zero and variance of 2.25 (nA)² to the input waveform. A peak detection algorithm is run (present in the scipy package in python), on the time-trace of the resulting neuron's membrane potentials to calculate the number of neural spikes. A simplistic definition of the firing rate for this simulation is used:

$$r = \frac{Total Number of Spikes}{1000 ms} \qquad ( 8 )$$

where:
r denotes the firing rate of the neuron model.

The firing rates for both neuron models were collected to construct the neural response rᵢ=[rᵢ₁,rᵢ₂]^{T} (rᵢ₁ for the Pyr neuron and rᵢ₂ for the PV neuron). The range of firing rates in the dataset for the Pyr neuron is [0 Hz, 40 Hz] and for the PV neuron is [0 Hz, 100 Hz].

Because the input to the model is the neural response r₁ and not the stimulus parameter \vec{θₜ}, the data is split into training, test and validation sets in the following manner: Split all possible neural responses present in [symbol] into training, validation and test firing rates. Let [symbol] be the sets containing the validation, training and test neural responses. Remove all the (\vec{θₜ},\vec{rₜ}) from the original dataset [symbol] where \vec{rₜ} is present in the test and validation set, to construct the training dataset D_{Tr}. Note that for any \vec{r} present in the test set or the validation set, there may be multiple stimulus parameter vectors \vec{θ} in the original dataset which produce \vec{r} and all of them are removed. The validation dataset [symbol] _{V} can be constructed similarly by removing (\vec{θₜ},\vec{rₜ}) from the original dataset [symbol] where \vec{rₜ} is present in the training set or the test set. For the test set, only the neural responses are stored (i.e., [symbol] _{Te}), to generate stimuli which produce those neural responses.

After training, each approach outputs a parameter \hat{θ} corresponding to every r. For the disclosed method, \vec{\hat{θ}} is the output of the estimated pseudoinverse, when provided with the input \vec{r}. For the first embodiment, \vec{\hat{θ}} is obtained as the output of the estimated pseudoinverse, when provided with the input \vec{r}. For the second embodiment (ensemble), the prediction scheme described above is used.

A figure of merit is used to evaluate the validation/test loss. The figure of merit is explained by taking an example of calculating it over the validation set. For every neural response \vec{r} present in [symbol] _{V}, obtain the corresponding \vec{\hat{θ}}, which is then fed to the neuron/model to obtain its actual firing rate \hat{r}^{act}. That is, g( [symbol] (\hat{r}))=\vec{r}^{act}.

Because there are m different neural responses, a NMAE is calculated for each individual neural response. Let the maximum and minimum values that can be achieved for j^{th} neural response be denoted as: rⱼ^{max} rⱼ^{min}, the NMAE is defined for the j^{th} neural response as:

$$NMAE = \frac{100}{{N}_{V} ( {r}_{j}^{max} - {r}_{j}^{min} )} {\sum }_{\vec{r} ∈ {ℛ}_{V}} \vert {r}_{j} - {r}_{j}^{act} \vert \qquad ( 9 )$$

where:
   |(·)| is the absolute function;
N_{V} is the number of response vectors present in the validation set; and
rⱼ and rⱼ^{act} are the j^{th} components of \vec{r} and \vec{r}^{act} respectively.

Note that for calculating NMAE, it is not necessary to explicitly know the restricted domain over which pseudoinverse has been estimated. The NMAE quantifies how close the neural response produced by the predicted stimulus is to the desired neural response on a scale of 0 to 100. The test NMAE can be calculated in a similar manner, where we replace the validation set by the test set. Notice that, for calculating the NMAE, access to the neuron is required (hence the need for accessing the neuron during hyper-parameter tuning.

Implementation—The details of one possible implementation of the disclosed method will now be discussed. To choose the hyper-parameters for all the data-driven approaches, the 10-fold cross-validation NMAE is used. Specifically, the hyper-parameter with the lowest cross-validation NMAE is picked. To calculate the 10-fold cross-validation NMAE, a 90%-10% split of the dataset is created, where 90% of the datapoints are in the training data and 10% of the datapoints are in the validation data, and the NMAE is calculated for the validation data. This process is repeated 10 times, and the average validation NMAE is used as the cross-validation NMAE. An upper and a lower bound of the range for each hyperparameter's search is obtained through a random search, followed by a grid search within these bounds.

Four different variants of the disclosed method were used, referred to herein as PATHFINDER-NET, PATHFINDER-NET-E, PATHFINDER-GP, and PATHFINDER-GP-E. In PATHFINDER-NET and PATHFINDER-GP, the inverse mapping ( [symbol] ) is estimated using a multi-layer perceptron (MLP) and Gaussian Process Regression, respectively. PATHFINDER-NET-E and PATHFINDER-GP-E are, respectively, the ensemble versions of PATHFINDER-NET and PATHFINDER-GP. To estimate the weight mapping (ŵ) for all 4 variants, a MLP is used. To implement the Gaussian Process Regression (GP) in PATHFINDER-GP and PATHFINDER-GP-E, an existing implementation was used with default parameters. In the toy examples, all of the MLPs used consist of 1-20 hidden layers, each with 10 hidden units, and GP's covariance kernel was chosen as a weighted summation of the Radial Basis Kernel, White Kernel and Dot Product Kernel. The weights for each kernel are automatically learned in GP by using cross-validation loss. For the neuron model, MLPs with 5 to 8 hidden layers were used, with each layer having 100 hidden units (although the hidden layers could have any number of hidden units), for the regressor (in PATHFINDER-NET and PATHFINDER-NET-E) and weight network for all the 4 variants. The Kernel for the neuron model case was the summation of the Radial Basis Kernel, White Kernel, Rational Quadratic and Dot Product Kernel. The output layer of the MLP for the regressor and the weight network has linear activation. For the toy examples, β was chosen as 0.001 for the cos 2πθ mapping, 0.001 for the

$${e}^{- \frac{{θ}^{2}}{2}}$$

mapping and 0.00001 for the (θ²−4)² mapping. For the neuron model the following values of β: 0.5, 0.1, and 0.05 were searched.

In addition to the data-driven methods, a brute force approach, akin to the 1-Nearest Neighbor Method, is used as a baseline method for the simulation study. In the baseline method, the training data is used as a look-up table, where the parameter vector present in the training dataset whose response vector is closest to the desired response vector under the L1 distance metric is output.

In one embodiment, Rectified Linear Unit (ReLU) activation is used for hidden layers of all approaches. MAF and MDN were trained by minimizing the negative log-likelihood (NLL). Alternatively, other activations, such as tanh, sigmoidal, exponential leaky unit and linear activation could be used.

Both the weight and the regressor were trained simultaneously by minimizing the loss in Eq. (2). During training, a simplex projection was performed on the batch output of the weight network to ensure the constraints specified in Eq. (2) are met). An Adam optimizer with a learning rate of 0.001 is used for minimizing the objectives for all techniques. Batch normalization was applied wherever applicable. Because all the methods were trained for multiple different sizes of the training datasets, batch size for Adam was chosen according to the training dataset size. Each model was trained until the validation loss converged (note loss instead of MAE), defined as a relative change of less than 0.1% in the validation loss between successive steps. Validation loss here was calculated using the more traditional method.

A potential reason for the data efficiency of the disclosed method is the maximization bias. The maximization bias refers to the phenomenon that the method tends to estimate the pseudoinverse with the largest number of datapoints. For example, in the cos(·) mapping, assume that the method only estimates 1 of the 6 pseudoinverses corresponding to the restricted domains [0, 0.5], [0.5, 1], [1, 1.5], [1.5, 2], [2, 2.5] and [2.5, 3]. Let nᵢ be the number of datapoints that lie in the restricted domain of the i^{th} pseudoinverse. Recall that the regularizer loss tries to give non-zero weights to as many datapoints as possible. Consequently, the regularizer encourages the choosing of the restricted domain with the largest number of datapoints (i.e., maxᵢ, nᵢ. For a size-n dataset sampled using a uniform distribution,

$$𝔼 [ {max}_{i} {n}_{i} ] = \frac{n}{6} + c \sqrt{n} ,$$

where [symbol] (·) is the expectation, and c is a constant). Note that, while the expected number of datapoints in any one restricted domain is n/6, the largest restricted domain has extra c\sqrt{H} datapoints. The method loss encourages inversion in just this restricted domain, and thus, is able to harness these extra datapoints, lowering the required overall sample size. On the other hand, the prior art methods, in one way or another, try to estimate the entire forward mapping, and are not designed to harness maximization bias.

The disclosed method is based on data-driven techniques for designing stimuli that produce desired neural responses. Traditionally, such stimuli are designed by choosing 1 or 2 “important” parameters (based on the intuition from the bio-physics of the system) and then brute-force searching across the 1 or 2 parameters to characterize the neural response. For example, one method considers the frequency of the sinusoidal stimulation as a parameter and brute-force searches across the frequency to characterize the neural response. A limitation of such brute-force search approaches is that they do not scale well as the number of parameters increases.

The most important advantage of the data-driven techniques is that allow exploration of a larger number of parameters. The Brute-force techniques require 250 datapoints to achieve the same performance as the disclosed method at 50 datapoints. Hence, the disclosed method allows an exploration of a larger number of parameters due to its data-efficiency. In practice, to an end-user, this implies that instead of choosing 1 or 2 most relevant parameters, they can now choose 5 or 10 most relevant parameters to search across. The advantage of exploring a larger number of parameters is that novel stimuli can be discovered which can produce neural responses which would be impossible to produce by just exploring 1 parameter. Intuitively, data-driven techniques are able to scale well to a higher number of parameters because they exploit the inherent smoothness of the neural response, in one form or other, and provide a more principled way of searching the parameter space.

The data-requirement for these data-driven techniques can be even further reduced by using adaptive sampling techniques. For example, the weight mapping of the disclosed method can be used for sampling only from the restricted domain thereby reducing the data requirements or the estimated p(\vec{θ}|\vec{r}) in the conditional density methods can be used to adaptively sample around a desired response \vec{r}_{des}. The characterization of the data-efficiency of the adaptive-sampling techniques is not addressed herein.

Disclosed herein is a data-driven method and model for designing stimuli to produce desired neural responses. Data-driven techniques allow end-users, such as clinicians and neuroscientists, to explore a larger number of parameters as compared to brute force search methods that allow only 1 or 2 parameter search space. Exploring larger number of parameters helps in designing novel stimuli that produce neural responses which are clinically relevant. The data-driven techniques helped in finding novel electrical waveforms for helping in treatment of Parkinsonian symptoms. It is desirable to make data-driven techniques as data-efficient as possible to increase their applicability in different neuromodulation contexts, as collecting data in neuromodulation domain is typically expensive. Towards increasing the data-efficiency of data-driven techniques, we the disclosed method is capable of designing stimuli for desired neural responses by estimating a pseudoinverse. In toy examples and neuron models, compared to existing data-driven techniques, namely MDN, MAF and NI, it was observed that the disclosed method requires the least amount of data for designing stimulus. Data-driven techniques provide a promising alternative to traditional brute-force search of parameters and can help in designing novel stimuli.

As would be realized by those of skill in the art, the specific examples discussed herein have been provided only as exemplary embodiments and the invention is not meant to be limited thereby. Modifications and variations are intended to be within the scope of the invention, which is given by the following claims:

1. A system comprising:
a first neural network or regression technique trained to estimate a pseudoinverse of a many-to-one forward mapping between a stimulus and a desired neuromodulation over a restricted domain; and
a second neural network trained to estimate weight mapping for the restricted domain;
wherein a loss function encourages the learning of a non-zero weight mapping only within the restricted domain over which the forward mapping is invertible; and
wherein a regularizer portion and a constraint encourages the learning of as large of a restricted domain as possible.

2. The system of claim 1 wherein the neural network comprises:
a first neural network to estimate a pseudoinverse; and
a second neural network to estimate the weight mapping.

3. The system of claim 1 wherein the restricted domain includes at least one set of parameters of a stimulus that produces the desired neuromodulation.

4. The system of claim 2 wherein only the parameters in the restricted domain have non-zero weights.

5. The system of claim 1 wherein the largest restricted domain is a largest domain over which the forward mapping can be inverted.

6. The system of claim 3 wherein the stimulus is an electrical waveform.

7. The system of claim 6 wherein the parameters are the amplitude, frequency and duration of the electrical waveform.

8. The system of claim 1 wherein the many-to-one forward mapping has a plurality of potential pseudoinverses and further wherein the system estimates one of the potential pseudoinverses.

9. The system of claim 7 wherein the system estimates both the pseudoinverses and the restricted domain.

10. The system of claim 2 wherein the first and second neural networks are multi-layer perceptron (MLP) networks.

11. The system of claim 10 wherein the first and second MLPs have 1-20 hidden layers.

12. The system of claim 11 wherein ReLU activation is used for the hidden layers.

13. The system of claim 1 wherein the regression techniques include Gaussian process regression, linear regression and polynomial regression.

14. A method comprising:
training a neural network to estimate a pseudoinverse of a many-to-one forward mapping between a stimulus and a desired neuromodulation over a restricted domain and to estimate a weight mapping for the restricted domain using a training dataset;
wherein a loss function encourages the learning of a non-zero weight mapping only within the restricted domain over which the forward mapping is invertible; and
wherein a regularizer portion and a constraint encourages the learning of as large of a restricted domain as possible.

15. The method of claim 14 further comprising:
estimating the pseudoinverse and the weight mapping.

16. The method of claim 14 further comprising:
estimating a pseudoinverse and a corresponding weight mapping of the restricted domain using the neural network;
identifying datapoints from a training dataset of the neural network that lie in the restricted domain using the estimated weight mapping;
removing the identified datapoints from the restricted domain to construct a new training dataset;
iterating the method until the training dataset is empty;
wherein the iteration results in a plurality of distinct pseudoinverses and corresponding restricted domains.

17. The method of claim 16 further comprising:
choosing a pseudoinverse from the plurality of pseudoinverses whose corresponding restricted domain contains a datapoint producing a response vector closest to a desired response of the forward mapping.
